(19) United States
(12) Patent Application Publication
(10) Pub. No.: US 2022/0133181 A1
(43) Pub. Date: May 5, 2022

(54) SYSTEMS, DEVICES, AND METHODS FOR DETECTING EARLY SHOCK

(71) Applicant: University of Washington, Seattle, WA (US)

(72) Inventors: Catherine Beni, Seattle, WA (US); Barclay Stewart, Seattle, WA (US); Jonathan Dov Posner, Seattle, WA (US); Aman Garg, Seattle, WA (US); Nelson Warner, Seattle, WA (US); Kenneth Shim, Seattle, WA (US); Yuan-Ni Yeh, Seattle, WA (US)

(21) Appl. No.: 17/514,941

(22) Filed: Oct. 29, 2021

Related U.S. Application Data
(60) Provisional application No. 63/108,204, filed on Oct. 30, 2020.

(51) Int. Cl. A61B 5/145; A61B 5/00; A61B 5/1473; G01N 33/68; G16H 40/67; G16H 10/40

(52) U.S. Cl. CPC A61B 5/14514; A61B 5/7282; A61B 5/0004; A61B 5/14546; A61B 2010/008; G01N 33/6893; G16H 40/67; G16H 10/40; A61B 5/14735

(57) ABSTRACT

Systems, devices, and methods to measure interstitial concentration of selected compounds over time to provide early detection of shock are described. In an example method, interstitial fluid is obtained from a subject. Analytes are detected in the interstitial fluid. At least one of the analytes corresponds to hypoxia of a tissue of the subject and at least one other analyte corresponds to vascular permeability of the subject. The example method further includes determining whether the subject is in shock based on a computing model and the detected analytes and generating an alert based on whether the subject is in shock. Treatments can be administered following the alert generation.

### CROSS-REFERENCE TO RELATED APPLICATION

This application claims the priority of U.S. Provisional Application No. 63/108,204, which was filed on Oct. 30, 2021 and is incorporated by reference herein in its entirety.

### STATEMENT REGARDING FEDERALLY SPONSORED RESEARCH

This invention was made with government support under Grant No. T32 GM121290, awarded by the National Institutes of Health. The government has certain rights in the invention.

### FIELD OF THE DISCLOSURE

The current disclosure describes systems, devices, and methods to measure the change in interstitial concentration of selected compounds over time to provide early detection of shock.

### BACKGROUND OF THE DISCLOSURE

Hemodynamic shock is a dangerous condition that occurs when an individual is unable to provide sufficient blood perfusion to their tissues. Hospitalized patients, particularly patients being treated in an intensive care unit (ICU) or other managed clinical setting can develop shock. In various cases, shock can be addressed by restoring blood perfusion. The symptoms of shock can be treated by administering intravenous fluids. If untreated, shock can lead to patient death.

In a clinical environment, shock can be difficult to identify. Although the clinical signs of shock are generally understood, these signs can be difficult to identify quickly in a clinical setting. In many clinical environments, a care provider is responsible for monitoring multiple patients simultaneously. Accordingly, the care provider may be delayed in identifying the early signs of shock in one patient due to their provision of care to another patient.

Furthermore, treatments for shock, such as intravenous fluid administration, can be harmful to the patient in some cases. For example, excess intravenous fluid administration can result in the accumulation of a proteinaceous fluid in peripheral tissues and organs, which can cause respiratory and/or renal failure. Thus, administering intravenous fluid to a patient who does not have shock or some other condition treatable by intravenous fluid may increase the patient's risk of receiving mechanical ventilation and dialysis, and may also reduce the patient's intestinal motility, nutritional absorption, and mobility, thereby increasing the patient's risk for extended rehabilitation after discharge and overall hospitalization. Thus, it is important to accurately diagnose shock prior to administering treatments.

There are limited bedside methods for rapid, precise, and robust evaluation of shock in the clinical environment. Further, no current devices exist for bedside measurement of capillary permeability, and physicians instead rely on indirect measures to evaluate the risk-benefit ratio of intravenous fluid administration and other treatments.

### BRIEF DESCRIPTION OF THE DRAWINGS

FIG. 1 illustrates an example environment for monitoring early shock in a clinical setting.

FIGS. 2A and 2B illustrate examples of probes that can be used to obtain samples of interstitial fluid from a patient. FIG. 2A illustrates an example of a linear probe inserted into the skin of a patient. FIG. 2B illustrates an example of a vertical probe inserted into the skin of a patient.

FIG. 3 illustrates an example exchange section that can be included in a vertical probe.

FIG. 4 illustrates examples of various detectors that can be used to detect the presence of different analytes in interstitial fluid of a patient.

FIG. 5 illustrates various electronic components within the detectors.

FIG. 6 illustrates an example of a monitoring device configured to be affixed to a thigh of a subject.

FIG. 7 illustrates an example process for detecting shock in a subject.

FIG. 8 illustrates an example process for detecting and reporting at least one analyte in interstitial fluid of a subject.

FIG. 9 illustrates an example of a computing device configured to perform various functionality described herein.

### DETAILED DESCRIPTION

The current disclosure describes systems, devices, and methods to measure the interstitial concentration of selected analytes over time to provide early detection of conditions (e.g., tissue hypoxia and capillary leakage) associated with the early stages of hemodynamic shock. Various implementations of the systems, devices, and methods described herein can enable early treatment of shock, prevention of unintended iatrogenic harm from excess intravenous (IV) fluid administration, and timely de-escalation. Ultimately, the systems, devices, and methods described herein could lead to reduced intensive care unit (ICU) length of stay, reduced dependency on mechanical ventilation, and overall improved patient outcomes.

Various implementations described herein relate to tools to aid physicians identify early shock and guide intravenous fluid therapy in critically ill patients. According to various examples, a device measures and assesses the temporal changes in concentrations of selected analytes in the peripheral tissues of a patient by sampling the interstitial fluid.

In particular implementations, the device is internally divided into multiple subsystems which perform different roles and functions. An example sample extraction subsystem is configured to obtain interstitial fluid samples from a patient via open flow microperfusion (OFM) probes. The sample extraction subsystem includes at least one probe, in various implementations. For example, the device includes a linear probe, a vertical probe, or a combination thereof. These types of probes differ on method of insertion and the mechanism behind extraction of interstitial fluid using perfusate. In various implementations, the device stores the perfusate in a bag or other type of receptacle. The perfusate, for instance, is an isotonic solution. The bag and/or other type of receptacle may be exchanged depending on the solution preference of the user. During operation of the device, the perfusate is run through the probe to enable exchange with the interstitial fluid. A fluid sample of interstitial fluid can be obtained by drawing the mixture of perfusate and interstitial fluid through the probe(s).

An example analyte measurement subsystem includes multiple lab-on-chip (LOC)-based microfluidic chips used to analyze the fluid sample. In various examples, the analyte measurement subsystem includes one or more LOCs that measure the concentrations of the analytes. In various examples, each analyte is measured with a separate LOC. For instance, each LOC may contain an enzyme-linked immunosorbent assay (ELISA) for a particular analyte-of-interest. Each LOC may be connected to one or more sensors and electrical components for data processing. In some cases, multiple LOCs operate in parallel to detect the respective analytes. In various examples, the analyte measurement subsystem further transmits data indicative of the detected analytes to a display subsystem. The analyte measurement subsystem, for instance, includes a transceiver. In some cases, the data is transmitted wirelessly (e.g., using BLUETOOTH™), by a wired connection, or a combination thereof. After passing through the LOCs, the fluid sample may be funneled to at least one waste receptacle (e.g., a collection vial) attached to the device. This vial can be removed and replaced.

An example display subsystem is configured to display concentrations of the analytes as measured by the analyte measurement subsystem. In some cases, the example display subsystem is part of the device itself and/or includes a standalone device (e.g., a monitor, a mobile device, or some other computing device) that is communicatively coupled to the device. In various implementations, the display subsystem also serves as the interface for the user to input desired parameters and control functionality of the device. For example, a user may use the display subsystem to input and/or adjust thresholds, the pump flow rate, number of measurement cycles, desired start time, analytes for detection, other parameters, or a combination thereof, which can be implemented by the device itself.

In some implementations, any of the subsystems can be omitted and/or one or more additional subsystems can be added to the device. The display subsystem, for instance, is configured to display one or more graphical user interface (GUI) elements indicative of the analytes detected by the analyte measurement subsystem. In some cases, the data is stored locally in the device or in a separate standalone device (e.g., a monitor) displaying both the current reading and a graphical representation of prior readings. The data is transmitted to the monitor either through cable connectors or wirelessly. In some examples, the device includes and/or is connected to an output device configured to output one or more signals indicative of the analytes detected by the analyte measurement subsystem.

In some cases, the device is connected to a wireless mobile interface configured to receive control signals from an external device, such as a mobile device. The device, for example, is configured to set and/or adjust thresholds, the pump flow rate, number of measurement cycles, desired start time, analytes for detection, or a combination thereof, based on the control signals, thereby enabling mobile control of the device. For instance, the device is communicatively coupled to an external computing device (e.g., a mobile device) through which a user can specify the operating characteristics of the device. In some specific examples, the flow rate can be selected from 0.1 uL/min-10 uL/min in increments of 0.1 uL/min. The number of measurement cycles can also be selected. For example, the user can specify that the device will measure the concentration of syndecan-1 in a patient three times during a time interval (e.g., 24 hours). The device may automatically calculate the sample volume needed for a single measurement cycle. In some examples, the time of the day for the measurement cycle (e.g., every day at 2 PM and 8 PM for the next three days) can be specified in advance, or the measurement cycle can be initiated instantaneously. In various cases, the analyte to be measured can be specified. In some examples, the device has preset parameters for specific analytes including sample volume, and flow rate.

The device may include one or more tubes that conduct various fluids described herein. For instance, the device includes at least one probe tube and at least one pump tube. The probe tube, for instance, has a 0.3-0.5 mm outer diameter (OD). The pump tube, for example, has a 0.3-0.4 mm OD. The tubes include, for example, medical-grade polytetrafluoroethylene (PTFE). External connectors on the device, for instance, include Luer lock connections which act as an interface between the internal pump and the external probe thereby allowing the connections between varying tube dimensions.

In various examples, the device is wearable. For example, the device is worn by the patient on a thigh, arm, or other body part while the patient is monitored in an ICU. In some cases, the device is reusable. For example, the device includes a replaceable cartridge that includes the LOC(s), perfusate, and waste fluids from previous analyses. Thus, when the device is transferred from one patient to another patient, the cartridge can be replaced to provide fresh LOC(s), perfusates, and an empty waste receptacle for the new patient. The probe may also be exchanged to provide a fresh probe for a new patient.

The device, for instance, is minimally invasive as the probe of the device is inserted into either skeletal muscle or a subcutaneous layer at a depth of no more than 1.00-1.50 mm below the surface of the patient's skin. In various examples, the patient can freely move the limb on which device is attached. The device, for example, refrains from causing undue tissue inflammation or additional injuries. In some cases, the device can be attached by making markings on the specific points by a surgical marker.

In various examples, the device is applied to the patient by a user. For instance, the skin at the insertion site is cooled prior to probe insertion. In some cases, the probe is inserted using a guide cannula. Thus, the device is relatively easy to insert. For example, a nurse or anyone trained to insert IV needles can apply the device to the patient. In some examples, the device includes an actuated needle that automatically inserts the probe, such that the device can be placed with minimal skill from the user.

Various implementations of the present disclosure can provide significant improvements to the technical field of patient monitoring. This disclosure describes examples of a device that enables point-of-care monitoring for shock with minimal user oversight. Unlike previous technologies, care providers can quickly, accurately, and minimally invasively monitor a patient for shock. Devices described herein can enable care providers to identify early shock before the patient experiences irreversible harm. Accordingly, implementations described herein can significantly improve patient outcomes and reduce care provider workloads.

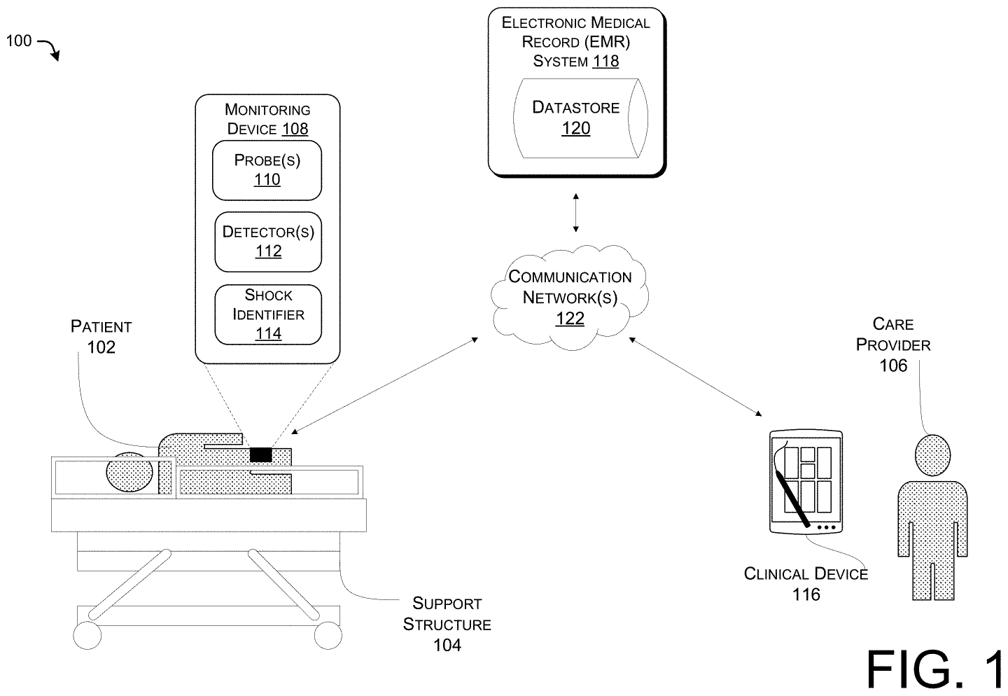

FIG. 1 illustrates an example environment 100 for monitoring early shock in a clinical setting. The environment 100 may include a patient 102 (e.g., a type of human subject). In some examples, the patient 102 is critically ill and monitored in an ICU of the clinical setting (e.g., a hospital). In some cases, the patient 102 is residing in the clinical setting for an extended period of time, such as hours, days, or weeks.

While in the clinical setting, the patient 102 may be resting on a support structure 104. The support structure 104 may be a gurney, a hospital bed, or some other structure configured to hold the patient 102 for an extended period of time.

The patient 102 may be monitored by a care provider 106 in the clinical setting. For instance, the care provider 106 may be a physician, a resident, a medical student, a physician's assistant, a nurse, a nursing assistant, a therapist, or some other clinical provider. Although not specifically illustrated in FIG. 1, the care provider 106 may be responsible for monitoring multiple patients (including the patient 102) in the clinical setting. For example, the care provider 106 may be responsible for the care of multiple critically ill patients in the ICU. Accordingly, the care provider 106 may be unable to constantly review the condition of the patient 102.

In various implementations, the patient 102 may be at risk for developing hemodynamic shock. As used herein, the terms “shock,” “hemodynamic shock,” and their equivalents, may refer to a medical condition in which the tissues of an individual receive insufficient oxygenation from blood circulation. That is, the oxygen demand of the tissues exceed the oxygen supplied to those tissues. Shock can have any number of root causes. For instance, the patient 102 may develop shock due to sepsis, a low volume of circulating blood (e.g., due to hemorrhage), excessive vomiting, diarrhea, burns, diabetes, myocardial infarction, cardiac arrhythmias, congestive heart failure, myocarditis, pulmonary embolism, arterial stenosis, anaphylaxis, a spinal cord injury, or any other condition that prevents sufficient blood circulation.

Shock progresses in multiple stages that increase in severity. In an initial stage, shock manifests as hypoxia, which causes an accumulation of pyruvate in the individual's tissues. The accumulated pyruvate is converted to lactate, which can result in lactic acidosis. Next, in a compensatory stage of shock, the individual may begin to hyperventilate in order to reduce the level of carbon dioxide in the blood, thereby reducing the acidity of the blood. The individual's body may release epinephrine and norepinephrine into the blood, which manifests as an increase in heart rate and blood pressure. Eventually, the individual may proceed to the progressive stage of shock. Hydrostatic pressure in the capillaries may increase due to blood accumulation, which can lead to fluid leakage into the tissues. The blood concentration and viscosity of the individual may increase. In examples wherein the bowel becomes ischemic, bacteria may enter the blood. Finally, shock can progress to a refractory stage. At this stage, the individual has experienced irreparable harm due to the shock. The individual's cells begin to die and the individual may experience permanent brain damage. Regardless of whether oxygenation is restored, the individual's adenosine stores are generally insufficient to restore enough ATP for the individual to restore normal tissue function.

In the example of FIG. 1, the care provider 106 may monitor the patient 102 for shock or other risky conditions while the patient 102 resides in the clinical setting. For example, the care provider 106 may periodically check the vital signs of the patient 102. However, it may be difficult for the care provider 106 to recognize the earliest stage of shock in the patient 102, because the corresponding physiological signs are sudden and subtle. Accordingly, the care provider 106 may be unable to recognize that the patient 102 is experiencing shock, and treat the shock of the patient 102, until the shock has progressed to the point of permanently damaging the patient 102.

In various implementations of the present disclosure, a monitoring device 108 accurately detects the signs of early shock in the patient 102. The monitoring device 108 can be affixed to the skin of the patient 102, such as attached to a leg, an arm, an extremity, an abdomen, or some other part of the patient 102. In some cases, the monitoring device 108 is portable. The monitoring device 108 may be implemented as a single-use, disposable patch. In various examples the overall size of the monitoring device 108 is relatively small (e.g., no larger than 10 cm by 10 cm). The monitoring device 108 may be configured to conform to a shape of a surface of the patient 102 without being limited by other injuries of the patient 102. In various examples, the monitoring device 108 is attached to the patient 102 by an adhesive, a glue, inbuilt Velcro tape, or the like.

The monitoring device 108 may include one or more probes 110 configured to extract interstitial fluid of the patient 102. As used herein, the term “interstitial fluid,” and its equivalents, may refer to a fluid present between cells of a multicellular organism. In various cases, interstitial fluid omits blood cells. According to various implementations, the probe(s) 110 may be part of a sample extraction subsystem configured to obtain interstitial fluid from the patient. The sample extraction subsystem, for instance, includes a pump system, perfusate, a probe, or any combination thereof. In some examples, the pump system includes multiple (e.g., 1, 2, 3, 4, 5, or greater) low flow-rate pumps that suction interstitial fluids from the patient. In various examples, pumps are designed to push the perfusate via OFM.

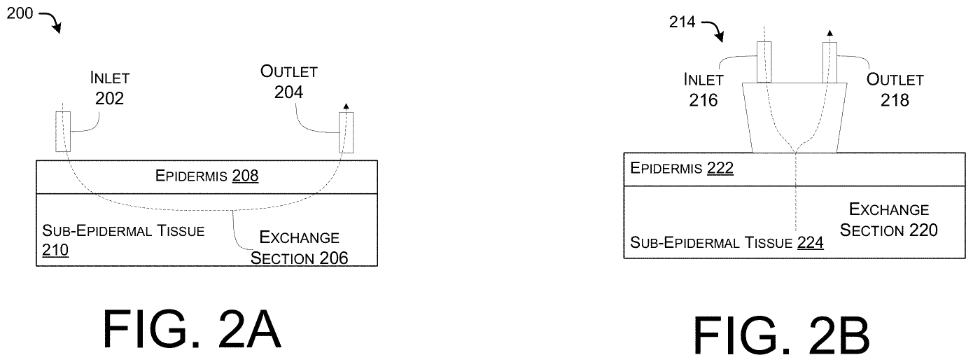

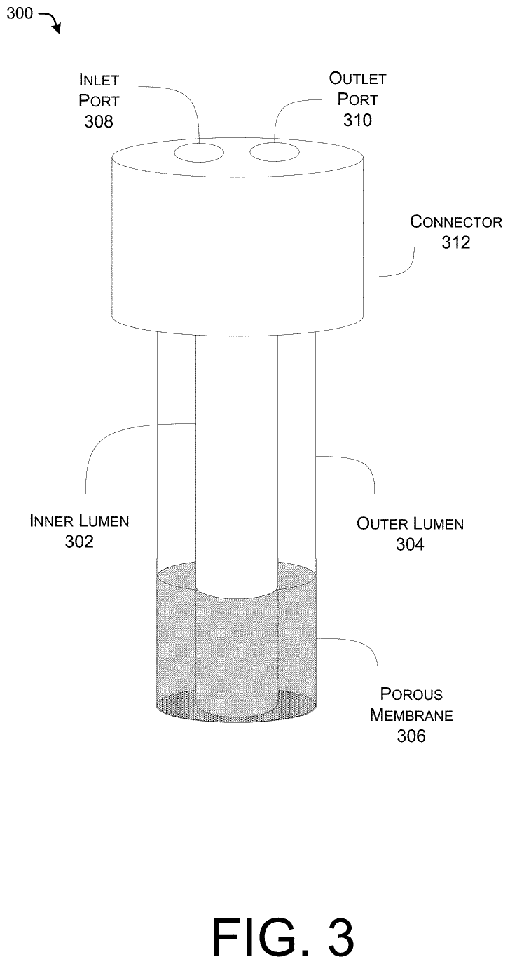

In various cases, the probe(s) 110 are configured to be at least partially inserted through the skin of the patient 102 and into the interstitium of the patient 102. The probe(s) 110 may be prevented from being inserted into the blood stream or other, deeper tissues of the patient 102. In some cases, the probe(s) 110 include at least one linear probe and/or at least one vertical probe. Examples of linear and vertical probes will be described below with reference to FIGS. 2A, 2B, and 3.

Unlike other types of technologies, the probe(s) 110 of the monitoring device 108 are configured to obtain interstitial fluid that includes proteins and other large molecules. For example, the probe(s) 110 may utilize OFM to extract samples of interstitial fluid from the patient 102. OFM is a minimally invasive in vivo interstitial fluid sampling technique. OFM is an alternative to microdialysis (MD) that is suited for sampling of large molecules. Instead of relying on a membrane, OFM uses probes with macroscopic openings to exchange substances in a liquid pathway between the inner cannula of the probe and the interstitial fluid. In various examples, convection-based fluid flows enable exchange between the interstitial fluids and the fluids within the probe of the monitoring device 108. For example, the monitoring device 108 uses a perfusate to obtain the interstitial fluid from the patient 102. The perfusate, for instance, includes an isotonic solution configured to capture one or more analytes-of-interest.

In various implementations, the probe(s) 110 may rely on a “push-pull mechanism” where a pre-selected fluid (e.g., the perfusate) is pushed through the probe(s) 110 from one end via a push pump. After exchange has occurred between the ISF and the perfusate, the resulting mixed fluid is pulled from the other end by a pull pump. In various examples of the present disclosure, the OFM performed by the probe(s) 110 and the corresponding pump(s) creates a uniform flow from one portion of a fluid circuit in the probe and/or device to another portion of the fluid circuit in monitoring device 108.

According to some implementations, the probe(s) 110 are removable from the rest of the monitoring device 108. For instance, the probe(s) 110 are connected to the rest of the monitoring device 108 through connectors (e.g., Luer lock connecters). In some cases, the probe(s) 110 are covered with a self-stabilizing adhesive and/or a plastic shield to protect the point of insertion into the patient 102 (e.g., an exchange section) and to secure the monitoring device 108 to the patient 102. These structures may prevent movement of the probe(s) 110 relative to the patient 102.

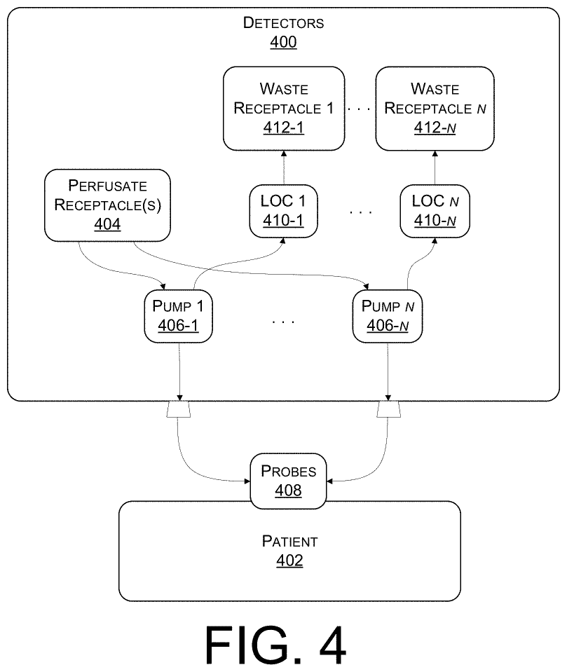

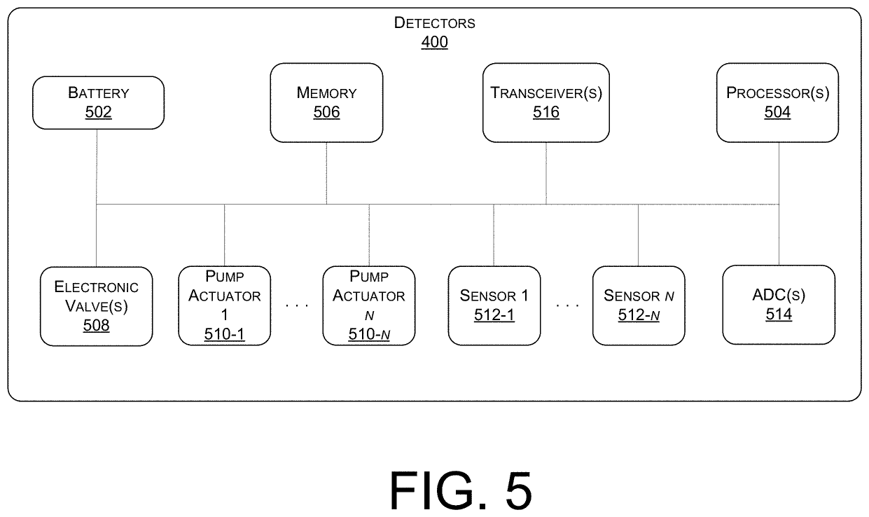

In addition, the monitoring device 108 may include one or more detectors 112 configured to detect the presence and/or concentration of one or more analytes in the interstitial fluid. Examples of the detector(s) 112 will be described below with reference to FIGS. 4 and 5. According to some examples, the analyte(s) include at least one protein. In some cases, the analyte(s) are associated with early shock. Early shock is associated with tissue hypoxia. As used herein, the term “hypoxia,” and its equivalents, may refer to a physiological state in which oxygen is not available at a tissue in sufficient amounts to support an aerobic metabolism of cells in the tissue. When a tissue is hypoxic, cells in that tissue transition from aerobic metabolism to anerobic metabolism, which includes a process of lactic acid fermentation. In various examples, the detector(s) 112 may detect the presence and/or concentration of at least one analyte associated with tissue hypoxia, such as lactate, pyruvate, succinate, or a combination thereof.

TABLE 1
Relationships between analytes associated
with tissue hypoxia and risk of shock

| | Relationship Between Analyte |
|---|---|
| | Amount in Interstitial Fluid |
| Analyte | and Risk of Shock |
| Lactate | Increased risk of shock |
| Pyruvate | Increased risk of shock |
| Succinate | Increased risk of shock |

In various examples, the initial stage of shock is associated with increased vascular (e.g., capillary) permeability, while shock resolution is associated with decreased vascular permeability. As used herein, the term “vascular permeability,” and its equivalents, can refer to a propensity of a blood vessel (e.g., a capillary) to leak contents (e.g., cells, ions, molecules, etc.) through the wall of the blood vessel. In some cases, the detector(s) 112 detect the presence and/or concentration of one or more markers associated with vascular permeability, such as syndecan-1, syndecan-4, alpha-1-microglobulin, beta-2-microglobulin, cystatin C, retinol-binding protein, albumin, inulin, creatinine, or a combination thereof. For example, Table 2 indicates the relationships between these analytes and the risk of the patient 102 for having developed shock.

TABLE 2
Relationships between analytes associated with
vascular permeability and risk of shock

| | Relationship Between Analyte |
|---|---|
| | Amount in Interstitial Fluid |
| Analyte | and Risk of Shock |
| Syndecan-1 | Increased risk of shock |
| Syndecan-4 | Increased risk of shock |
| Alpha-1-Microglobulin | Increased risk of shock |
| Beta-2-Microglobulin | Increased risk of shock |
| Cystatin C | Increased risk of shock |
| Retinol-Binding Protein | Increased risk of shock |
| Albumin | Increased risk of shock |
| Inulin | Increased risk of shock |
| Creatinine | Increased risk of shock |

Shock can also be associated with inflammation, coagulopathy, and the presence of one or more electrolytes. In some implementations, the detector(s) 112 detect one or more markers associated with inflammation and endothelial integrity, such as soluble thrombomodulin, e-selectin, soluble intercellular adhesion molecule 1 (ICAM-1), soluble endothelial-leukocyte adhesion molecule 1 (ELAM-1), soluble vascular cell adhesion protein 1 (VCAM-1), interleukin 6 (IL-6), procalcitonin, or a combination thereof. Table 3 indicates the relationships between these analytes and the risk of the patient 102 for having developed shock.

TABLE 3
Relationships between analytes associated with inflammation
and endothelial integrity and risk of shock

| | Relationship Between Analyte |
|---|---|
| | Amount in Interstitial Fluid |
| Analyte | and Risk of Shock |
| Thrombomodulin | Increased risk of shock |
| E-selectin | Increased risk of shock |
| ICAM-1 | Increased risk of shock |
| ELAM-1 | Increased risk of shock |
| VCAM-1 | Increased risk of shock |
| IL-6 | Increased risk of shock |
| Procalcitonin | Increased risk of shock |

In some examples, the detector(s) 112 detect one or more markers associated with coagulopathy, such as antithrombin III, protein C, protein S, fibrinogen, tissue plasminogen activator (TPA), alpha 2-antiplasmin, beta-thromboglobulin, platelet factor 4, or a combination thereof. Table 4 indicates the relationships between these analytes and the risk of the patient 102 for having developed shock.

TABLE 4
Relationships between analytes associated
with coagulopathy and risk of shock

| | Relationship Between Analyte |
|---|---|
| | Amount in Interstitial Fluid |
| Analyte | and Risk of Shock |
| Antithrombin III | Increased risk of shock |
| Protein C | Increased risk of shock |
| Protein S | Increased risk of shock |
| Fibrinogen | Increased risk of shock |
| TPA | Increased risk of shock |
| Alpha 2 antiplasmin | Increased risk of shock |
| Beta-thromoglobulin | Increased risk of shock |
| Platelet factor 4 | Increased risk of shock |

In some cases, the detector(s) 112 detect one or more electrolytes or other analytes, such as sodium, potassium, bicarbonate, chloride, glycerol, or a combination thereof. Table 5 indicates the relationships between these analytes and the risk of the patient 102 for having developed shock.

TABLE 5
Relationships between electrolyte analytes and risk of shock

| | Relationship Between Analyte |
|---|---|
| | Amount in Interstitial Fluid |
| Analyte | and Risk of Shock |
| Sodium | Increased risk of shock |
| Potassium | Increased risk of shock |
| Bicarbonate | Increased risk of shock |
| Chloride | Increased risk of shock |
| Glycerol | Increased risk of shock |

In various implementations, shock severity is correlated to the concentration of one or more analytes in the interstitial fluid and/or blood. For example, the concentration of an analyte associated with an increased risk of shock may be positively correlated to shock severity. Accordingly, the severity of shock can be determined based on the analyte(s) detected by the detector(s) 112.

In some cases, the detector(s) 112 detect endogenous compounds that rapidly equilibrate between the interstitial space and the blood, such as urea and glucose. The presence and/or concentration of these compounds may be used as reference values from which the concentration measurement of other listed analytes can be interpreted. For example, the detector(s) 112 may detect a concentration of glucose in the interstitial fluid, and a separate device may detect a concentration of glucose in the blood. Because the concentration of glucose may be assumed to be the same in the interstitial fluid and the blood, the detected concentrations of glucose in the interstitial fluid and the blood may be used to calibrate and determine the blood concentration of other analytes detected in the interstitial fluid.

In certain examples, the patient 102 is determined to be in shock by comparing one or more analytes to one or more thresholds (also referred to as “baselines”). For instance, the analytes may be compared to a “healthy” or “normal” baseline. Healthy or normal baseline levels can be derived from reference populations that do not have shock or a condition associated with shock. Alternatively, the patient 102 can be identified as having shock if an amount of at least one of the analyte-of-interest in the interstitial fluid of the patient 102 does not significantly differ from a reference level within a 95% confidence interval, wherein the reference level is based on the amount of the at least one analyte-of-interest in another subject with confirmed shock. In particular implementations, a “baseline” or “reference level” can refer to a standardized value for any analyte described herein which represents a level not associated with shock (baseline) or a level associated with a particular type of shock (reference level).

As is understood by one of ordinary skill in the art, determining a baseline amount of an analyte for healthy patients can vary based on the assay used. Standard baseline (or reference) levels can vary from source to source or from laboratory to laboratory. In particular embodiments, the interstitial fluid of healthy patients have a baseline level of an example analyte. Examples of baseline ranges (e.g., upper and lower baselines) for various example analytes are listed below in Table 6.

TABLE 6
Baseline ranges for example analytes-of-interest

| Analyte | Baseline | |
|---|---|---|
| Glucose | 100-140 | mg/dL |
| Sodium | 135-145 | mEq/L |
| Potassium | 3.6-5.2 | mEq/L |
| Chloride | 96-106 | mEq/L |
| Glycerol | 160-290 | uM |
| Lactate | 0.5-2 | mM |

A “dataset” as used herein is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Datasets can be used by an interpretation function to derive a likelihood that a subject is experiencing shock, which can provide a quantitative measure of shock when compared to a baseline or reference level.

In various examples, a determination of shock of the patient 102 is made against a non-personal baseline (healthy) or reference level (shock). In certain examples, human subjects with shock have an elevated analyte profile. An elevated analyte profile can include elevated expression of any of the analytes described herein. In certain examples, elevated means above a healthy threshold within a particular assay. In particular embodiments, human subjects with shock can have a reference analyte level.

As indicated, a level of at least one analyte in the interstitial fluid of the patient 102 can be assessed. These molecules can be measured according to any method known in the art, such as enzyme-linked immunosorbent assay (ELISA), direct enzyme immunoassay, electrochemiluminescence via sandwich assay, western blot analysis, dot blot, northern blot analysis, nuclease protection assay, in situ hybridization, or polymerase chain reaction (PCR).

In various implementations, the detector(s) 112 are configured to detect the amounts of different types of analytes. For example, the detector(s) 112 may be configured to detect at least one first analyte associated with tissue hypoxia and at least one second analyte associated with vascular permeability. By assessing multiple facets of the condition of the patient 102, the monitoring device 108 may determine whether the patient 102 has developed shock with increased accuracy.

Each detector 112 may include multiple structural elements for detecting the presence and/or concentration of the analyte(s). In some cases, the detector(s) 112 include tubing, channels (e.g., microfluidic channels), or other mechanisms for transporting fluid between elements of the monitoring device 108.

In some cases, the detector(s) 112 include one or more pumps (e.g., peristaltic pumps) configured to propel the interstitial fluid and/or reagents throughout the monitoring device 108. In particular implementations, each pump generates a flow rate of the interstitial fluid through the monitoring device 108 that is between 0.1-10 μL/min. The flow rate may be continuous and/or initiated at discrete time intervals. According to particular examples, the flow rate through one of the detector(s) 112 is dependent on the type of analyte being analyzed and the type of perfusate selected. Different analytes may have different optimal flow rates. Thus, if the detector(s) 112 are configured to analyze multiple analytes, the detector(s) 112 may include pumps that are configured to apply different flow rates for respective analytes-of-interest.

In various implementations, the detector(s) 112 include one or more LOCs that are configured to detect the analyte(s) in the interstitial fluid obtained by the probe(s) 110. In some examples, the detector(s) 112 include multiple microfluidic chips arranged in parallel. Each LOC, for instance, contains an ELISA for a particular analyte-of-interest. In various examples, each analyte is measured with a separate LOC within the detector(s) 112. Each LOC may be connected to one or more sensors and electrical components for data processing. In some cases, multiple LOCs operate in parallel to detect the respective analytes. After passing through the LOCs, the fluid sample can be funneled to at least one waste receptacle (e.g., a collection vial, waste bag, etc.) within the monitoring device 108. This waste receptacle can be removed and replaced, such as by a user.

According to some examples, the detector(s) 112 are configured to detect different analytes at different sampling rates or in response to different types of events. For example, a first analyte may be detected by a first LOC at a first sampling rate and a second analyte may be detected by a second LOC at a second sampling rate. In some implementations, a third analyte is detected in response to an event, such as the first analyte being detected greater than a predetermined threshold or in response to a signal from an external device.

The monitoring device 108 may also include a shock identifier 114. The shock identifier 114 may be implemented in hardware, software, or a combination thereof. The shock identifier 114, in various cases, is configured to detect whether the patient 102 is in shock based on the presence and/or concentration of the analyte(s) in the interstitial fluid. According to some examples, the shock identifier 114 compares an amount of each analyte to at least one respective threshold. For example, the shock identifier 114 may determine that the patient 102 is suspected of being in early shock if an analyte in the interstitial fluid of the patient 102 is greater than a first threshold or lower than a second threshold. In some implementations, the shock identifier 114 identifies that the patient 102 is suspected of having early shock based on a combination of multiple analytes. For instance, the shock identifier 114 may determine that the patient 102 is suspected of being in early shock if a first analyte is greater than the first threshold or lower than the second threshold and if a second analyte is greater than a third threshold or lower than a fourth threshold, but may not determine that the patient is suspected of being in early shock if the first analyte is greater than the first threshold or lower than the threshold and if the second analyte is lower than the third threshold or greater than the fourth threshold.

In some cases, the shock identifier 114 determines a shock severity of the patient 102 based on the concentration of the detected analyte(s) in the interstitial fluid and/or blood. For example, if the shock identifier 114 determines that the concentration of an example analyte is greater than a lower threshold but less than an upper threshold, the shock identifier 114 may determine that the patient 102 has a relatively low shock severity. However, if the shock identifier 114 determines that the concentration of the example analyte is greater than both the lower threshold and the upper threshold, the shock identifier 114 may determine that the patient 102 has a relatively high shock severity.

In some implementations, the monitoring device 108 includes an external housing that at least partially encloses one or more constituents of the monitoring device 108. The housing includes a material that does not absorb moisture and is resistant to bacterial accumulation, which can provide comfort to the patient 102 without substantial skin damage. For example, the external housing of the monitoring device 108 includes (medical-grade) polyethylene or some other polymer.

In some examples, the monitoring device 108 is battery-powered. For instance, the monitoring device 108 may include a lithium-ion battery that can be charged and recharged (e.g., by plugging the monitoring device 108 into wall current, a 5 Volt (V) port, etc.). The battery, for instance, can provide a life of at least 12 hours or at least 100 measurement cycles. In some cases, the battery provides a smaller or greater life or number of measurement cycles.

In various implementations, the monitoring device 108 includes an output device configured to output an indication of the detected analyte(s) and/or whether the patient 102 is experiencing shock. For example, the monitoring device 108 includes a display screen that visually outputs the concentration of the analyte(s). Upon detecting that the patient 102 is suspected of experiencing shock, the monitoring device 108 may visually output an alert (e.g., text, shapes, specific colors, blinking lights, etc.) that indicate the patient 102 is potentially experiencing shock. According to some examples, the monitoring device 108 may output an alert indicating the severity of the shock. In some cases, the monitoring device 108 includes a speaker that audibly outputs an alert indicating that the patient 102 is suspected of experiencing shock.

In some cases, the monitoring device 108 transmits a signal to an external clinical device 116 indicating the concentration of the analyte(s), the presence of the analyte(s), whether the patient 102 is experiencing shock, or a combination thereof. In some implementations, the monitoring device 108 outputs the concentration of the analyte(s), the presence of the analyte(s), whether the patient 102 is experiencing shock, or any combination thereof. In some implementations, the monitoring device 108 outputs an alert to the care provider 106 if the patient 102 is suspected of experiencing shock. For example, the clinical device 116 may output the alert visually using a display screen, audibly using a speaker, or by vibrating (e.g., using a haptic feedback device integrated into the clinical device 116). In various implementations, the care provider 106 can check the condition of the patient 102 upon discerning the alert.

In various implementations, the shock of the patient 102 is treated by the care provider 106 or the monitoring device 108 itself. For example, upon receiving the alert, the care provider 106 may administer various treatments, including intravenous fluids, antibiotics, and blood products, to the patient 102 to treat the shock. In some cases, the care provider 106 may perform further diagnostic tests on the patient 102 to determine the reason why the patient 102 is experiencing shock. The care provider 106 may treat the cause of the shock in the patient 102. For example, the care provider 106 may determine that the patient 102 is experiencing internal bleeding and may perform a surgical procedure to halt the internal bleeding. In some cases, the monitoring device 108 or some other device administers intravenous fluids to the patient 102.

In various examples, the monitoring device 108 transmits a signal to an electronic medical record (EMR) system 122 indicating the concentration of the analyte(s), the presence of the analyte(s), whether the patient 102 is experiencing shock, or a combination thereof. The EMR system 122 may include a datastore 120 that includes an EMR of the patient 102. In various cases, the EMR system 122 may update the EMR of the patient 102 based on the signal from the monitoring device 108. For example, the EMR system 122 may store, in the EMR of the patient 102, an indication of the time at which the analyte(s) of the patient 102 were measured and/or when shock was detected in the patient 102.

The monitoring device 108 may be configured to communicate with the clinical device 116 and the EMR system 118 over one or more communication networks 122. The communication network(s) 112 may include at least one wired interface, at least one wireless interface, or a combination thereof. Examples of wired interfaces include Ethernet cables, electrical cables, optical cables, and the like. Examples of wireless interfaces include BLUETOOTH™ interfaces, Institute of Electrical and Electronics (IEEE) interfaces (e.g., WI-FI™ interfaces), 3rd Generation Partnership Project (3GPP) interfaces (e.g., Long Term Evolution (LTE) radio interfaces, New Radio (NR) radio interfaces, etc.), Near Field Communication (NFC) interfaces, and the like. In some implementations, the communication network(s) 112 include at least one wide area network (WAN), such as the Internet. The communication network(s) 112 may include at least one core network, such as an Internet Protocol (IP) Multimedia Subsystem (IMS) network and/or a cellular core network (e.g., an Evolved Packet Core (EPC), 5^{th} Generation (5G) Core (5GC), etc.). Electrical signals, electromagnetic signals, optical signals, and the like, may encode data and be transmitted over one or more interfaces in the communication network(s) 112 by various elements within the environment 100.

According to various implementations, the monitoring device 108 may receive signals from the clinical device 116 and/or the EMR system 118. In some cases, the care provider 106 may input, into the clinical device 116, a sampling frequency for the patient 102. For instance, the care provider 106 may specify a relatively high sampling frequency if the care provider 106 determines that the patient 102 is at a heightened risk for developing shock. The clinical device 116 may transmit, to the monitoring device 108, a signal indicating the sampling frequency. Based on the signal, the monitoring device 108 may detect whether the patient 102 has early shock at the sampling frequency. In various cases, the EMR system 118 may transmit a signal to the monitoring device 108 indicating the sampling frequency or a condition (e.g., a recent surgery) indicated in the EMR of the patient 102 that impacts the risk of the patient 102 for developing shock. Based on the signal from the EMR system 118, the monitoring device 108 may detect whether the patient 102 has early shock at the specified sampling frequency, or may set the sampling frequency based on the condition of the patient 102. In various cases, the shock identifier 114 of the monitoring device 108 may adjust a threshold, sampling frequency, or other condition for detecting shock in the patient 102 based on the signals received from the clinical device 116 and/or the EMR system 118.

According to various examples, the monitoring device 108 includes electrical ports. For instance, the monitoring device 108 includes standard electrical ports (e.g., a Universal Serial Bus (USB) port, a micro-USB port, a High-Definition Multimedia Interface (HDMI) port, etc.), for instance. A cord (e.g., a USB cord, a micro-USB cord, an HDMI cord, etc.) can be plugged into one of the ports of the monitoring device 108 and a port of an external device, such as the clinical device 116. In various examples, the clinical device 116 is configured to output various parameters, such as instantaneous and/or temporal variation in concentrations of analytes-of-interest. For example, the clinical device 16 includes a display that is configured to visually output the parameters.

In various implementations, the monitoring device 108 receives calibration data that enables the monitoring device 108 to calculate the amount of each analyte-of-interest detected in the interstitial fluid. In some instances, a separate device may identify the amount of a given analyte-of-interest in the blood of the patient 102. For example, the care provider 106 may enter an amount (e.g., concentration) of a first analyte (e.g., glucose) detected in the blood of the patient into the clinical device 116 and/or the datastore 120 of the EMR 118. In some cases, a separate device may detect the amount of the first analyte in a blood sample obtained from the patient 102. The clinical device 116 and/or the EMR system 118 may transmit, over the communication network(s) 112, data indicating the amount of the first analyte detected in the blood of the patient 102. In various cases, the amount of the first analyte detected in the blood of the patient may be equal to, or at least correlated with, the amount of the first analyte present in the interstitial fluid of the patient 102. The detector(s) 112 may detect a first signal indicative of the amount of the first analyte and a second signal indicative of an amount of a second analyte in the interstitial fluid sample. The shock identifier 114 may use the amount of the first analyte detected in the blood of the patient to determine the amount of the second analyte in the interstitial fluid sample. For example, the shock identifier 114 may determine a scalar quantity a, wherein a magnitude of the first signal multiplied by a equals the amount of the first analyte detected in the blood of the patient. The shock identifier 114 may then infer that a magnitude of the second signal multiplied by a equals the amount of the second analyte present in the blood of the patient 102. Accordingly, the monitoring device 108 may calculate and output (e.g., to a user, to the clinical device, to the EMR system 118, etc.) the amount of the second analyte present in the blood of the patient 102.

FIGS. 2A and 2B illustrate examples of probes that can be used to obtain samples of interstitial fluid from a patient. FIG. 2A illustrates an example of a linear probe 200 inserted into the skin of a patient. As shown, the linear probe 200 includes an inlet 202, an outlet 204, and an exchange section 206. The inlet 202 receives perfusate from a receptacle in a monitoring device (e.g., the monitoring device 108 described above with reference to FIG. 1). In various implementations, the device stores the perfusate in a bag or other receptacle. The perfusate, for instance, is an isotonic solution.

The perfusate flows from the inlet 202 to the exchange section 206. The exchange section 206 may be disposed through an epidermis 208 of the patient and in a sub-epidermal tissue 210 of the patient. The sub-epidermal tissue 212 may include interstitial fluid of the patient. For example, the sub-epidermal tissue 202 may include dermis of the patient, interstitium of the patient, some other soft tissue that includes interstitial fluid of the patient, or a combination thereof. In various implementations, the sub-epidermal tissue 202 omits vasculature of the patient, such that the linear probe 202 is not in contact with the bloodstream of the patient.

The exchange section 206 may be a porous conduit through which the perfusate can flow from the inlet 202 and through which a mixture of the perfusate and the interstitial fluid can flow to the outlet 204. The exchange section 206 of the linear probe 202 may be cross the epidermis 208 of the patient at two locations. In some implementations, the exchange section 206 is an open exchange area that can perform OFM. The exchange section 206 may include pores or a mesh with openings that are sufficiently large enough to pass proteins and other macromolecules from the interstitial fluid into the exchange section 206. For example, the openings may have widths in a range of 0.1 mm to 0.5 mm. In various implementations, the exchange section 206 includes a polymer and/or a metal. The inlet 202 and/or the outlet 204 may include tubing, such as polystyrene tubing.

The linear probe 200, for example, is inserted horizontally into tissue. In some examples, the linear probe 200 is inserted at a depth of 1.00-3.00 mm below the surface of epidermis 222. The exchange section 206 may include a braided stent with pores. In a particular example, the shaft length of the linear probe 200 is 200.00 mm; the internal diameter of the exchange section 206 is 0.40 mm; the outer diameter of the exchange section 206 is 0.55 mm; the length of the exchange section 206 is 15.00 mm; and the pores have 0.10 mm diameters.

FIG. 2B illustrates an example of a vertical probe 214 inserted into the skin of a patient. As shown, the vertical probe 214 includes an inlet 216, an outlet 218, and an exchange section 220. The inlet 216 receives perfusate from a receptacle in a monitoring device (e.g., the monitoring device 108 described above with reference to FIG. 1). In various implementations, the device stores the perfusate in a bag or other receptacle. The perfusate, for instance, is an isotonic solution.

The perfusate flows from the inlet 216 to the exchange section 220. The exchange section 220 may be disposed through an epidermis 222 of the patient and in a sub-epidermal tissue 224 of the patient. The sub-epidermal tissue 224 may include interstitial fluid of the patient. For example, the sub-epidermal tissue 202 may include dermis of the patient, interstitium of the patient, some other soft tissue that includes interstitial fluid of the patient, or a combination thereof. In various implementations, the sub-epidermal tissue 224 omits vasculature of the patient, such that the vertical probe 214 is not in contact with the bloodstream of the patient.

The exchange section 220 may be a conduit through which the perfusate flows from the inlet 216 into the sub-epidermal tissue 224 and through which a mixture of perfusate and interstitial fluid flows into the outlet 218. In some implementations, the exchange section 220 includes a dual-lumen needle that includes an inner lumen and an outer lumen that is disposed around the inner lumen. In some cases, perfusate flows from the inlet 216 through the inner lumen of the exchange section 220 and a mixture of perfusate and interstitial fluid flows between the inner lumen and the outer lumen into the outlet 218, or vice versa. Accordingly, a sample of the interstitial fluid (including proteins and other macromolecules within the interstitial fluid) can be captured using the vertical probe 214. In some cases, the vertical probe 214 includes a polymer and/or a metal. The inlet 216 and the outlet 218 can include tubing, such as polystyrene tubing.

In some cases, the vertical probe 214 is inserted at a greater depth than the linear probe 200. For example, the vertical probe 214 is inserted at a depth of 0.500-1.000 cm from the surface of the epidermis 222. In a particular example, the shaft length of the vertical probe 214 is 8.00 mm; a length of the exchange section 220 is 1.00 mm; a total length of the vertical probe 214 is 9.00 mm; the pores have 0.05-0.10 mm diameters; and the vertical probe 214 has a diameter of 0.50 mm.

FIG. 3 illustrates an example exchange section 300 that can be included in a vertical probe. As shown, the exchange section 300 includes an inner lumen 302 and an outer lumen 304. The inner lumen 302 is disposed inside of the outer lumen 304. In some implementations, a tip of the outer lumen 304 is sharp, such that the exchange section 300 can pierce the epidermis of a patient. The inner lumen 302 and/or the outer lumen 304 can include a metal, such as stainless steel. In various implementations, the tip of the outer lumen 304 is covered by a porous membrane 306. The porous membrane 306, for example, includes pores that are sufficiently large to pass macromolecules (e.g., proteins) within the interstitial fluid of the patient. For example, the pores have widths in a range of 0.1 mm to 0.5 mm.

During operation, an inlet port 308 receives perfusate from another component of a monitoring device. Due to a positive pressure differential between the inlet port 308 and the inner lumen 302, the perfusate is pushed down through the inner lumen 304 and through the porous membrane 306. The perfusate, for example, may enter a sub-epidermal tissue of the patient that includes interstitial fluid of the patient. A negative pressure differential is generated between the outlet port 310 and a space between the inner lumen 304 and the outer lumen 304, which causes a mixture of the perfusate and the interstitial fluid to be pulled through the porous membrane 306 and up into the outlet port 310. Accordingly, a sample of the interstitial fluid can be obtained. In some alternate implementations, the sample is pulled up through the inner lumen 302 and the perfusate is pushed out through the space between the inner lumen 302 and the outer lumen 304.

The exchange section 300 may further include a connector 312. The connector 312 may fluidly couple the inner lumen 302 and the outer lumen 304 to the inlet port 308 and the outlet port 310. For example, the connector 312 may include a Luer-lock connection.

FIG. 4 illustrates examples of various detectors 400 that can be used to detect the presence of different analytes in interstitial fluid of a patient 402. For example, the detectors 400 may be integrated into a monitoring device, such as the monitoring device 108 described above with reference to FIG. 1.

The detectors 400 may include one or more perfusate receptacles 404 that store perfusate. The perfusate receptacle(s) 404 may include vials, bags, or any other type of structure that is configured to hold fluids, such as perfusate. In some examples, the vials, bags, and other structures can be removed, refilled, and/or emptied manually by a user. The perfusate, for example, may include water, sodium, chloride, lactate, glucose, or a combination thereof. For example, the perfusate may be an isotonic solution.

The detectors 400 may further include pump 1 to n 406-1 to 406-n, wherein n is a positive integer. Each of the n pumps may be configured to pump the perfusate from the perfusate receptacle(s) 404 to probes 408 that are attached to the patient 402. In some implementations, the probes 408 include n probes that are respectively coupled to pumps 1 to n 406-1 to 406-n. For example, pump 1 406-1 may be configured to push perfusate into a first one of the probes 408 and pump n may be configured to push perfusate into an nth one of the probes 408. Pump 1 to n 406-1 to 406-n may include peristaltic pumps.

According to various implementations, pump 1 to n 406-1 to 406-n are further configured to pull samples of interstitial fluid from the patient 402, into the probes 408, and up to LOCs 1 to n 410-1 to 410-n. For example, the pumps 406-1 to 406-n may be coupled to respective valves that selectively prevent the perfusate from being pushed into the LOCs 410-1 to 410-n and selectively prevent the interstitial fluid samples from being pulled up into the perfusate receptacle(s) 404. The LOCs 1 to n 410-1 to 410-n are configured to respectively detect n analytes in the interstitial fluid samples. For instance, LOC 1 410-1 may be configured to detect an analyte associated with hypoxia of a tissue of the patient 402 and LOC n 410-n may be configured to detect an analyte associated with vascular permeability of the patient 402.

The LOCs 1 to n 410-1 to 410-n may include fluid circuits configured to detect the analytes. For example, the LOCs 1 to n 410-1 to 410-n may include microfluidic circuits configured to respectively detect the n analytes. As used herein, the term “microfluidic circuit,” and its equivalents, may refer to a device that includes multiple channels, valves, chambers, and other elements configured to hold and/or move microscale (e.g., 1 microliter (μL) to 1,000 μL) amounts of at least one fluid (e.g., a liquid). In some cases, the LOCs 1 to n 410-1 to 410-n are composed of silicone, silicon, glass, one or more polymers (e.g., polydimethylsiloxane (PDMS), poly(methylmethacrylate) (PMMA), polycarbonates (PC), etc.), or any combination thereof. The LOCs 1 to n 410-1 to 410-n may include various channels that transport reagents and interstitial fluid samples to and from various reaction chambers within the LOCs 1 to n 410-1 to 410-n. In some implementations, the LOCs 1 to n 410-1 to 410-n include one or more receptacles that store reagents for performing chemical reactions involving the interstitial fluid samples. Further, in some cases, the LOCs 1 to n 410-1 to 410-n include one or more pumps and/or valves configured to direct the flow of the reagents and interstitial fluid samples throughout the LOCs 1 to n 410-1 to 410-n.

According to some examples, the LOCs 1 to n 410-1 to 410-n are configured to detect the analytes by performing ELISA tests on the interstitial fluid samples. The LOCs 1 to n 410-1 to 410-n may include vials, bags, or any other type of structure that is configured to hold fluids, such as reagents. In some examples, the vials, bags, and other structures can be removed, refilled, and/or emptied manually by a user. As used herein, the term “reagent,” and its equivalents, may refer to a substance that is consumed or otherwise involved in a chemical reaction. For example, various reagents described herein include binding domains that specifically bind to analytes-of-interest. In particular implementations, binding domains include antibodies, antibody binding domains, peptides, peptide aptamers, nucleic acids, nucleic acid aptamers, spiegelmers, or combinations thereof.

“Antibodies” are one example of binding domains and include whole antibodies or binding fragments of an antibody, e.g., Fv, Fab, Fab′, F(ab′)2, Fc, and single chain Fv fragments (scFvs) or any other effective binding fragments of an immunoglobulin. Antibodies or antigen binding fragments include all or a portion of polyclonal antibodies, monoclonal antibodies, human antibodies, humanized antibodies, synthetic antibodies, chimeric antibodies, bispecific antibodies, mini bodies, and linear antibodies.

Peptide aptamers include a peptide loop (which is specific for a target protein) attached at both ends to a protein scaffold. This double structural constraint greatly increases the binding affinity of the peptide aptamer to levels comparable to an antibody. The variable loop length is typically 8 to 20 amino acids (e.g., 8 to 12 amino acids), and the scaffold may be any protein which is stable, soluble, small, and non-toxic. Peptide aptamer selection can be made using different systems, such as the yeast two-hybrid system (e.g., Gal4 yeast-two-hybrid system) or the LexA interaction trap system.

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. In particular embodiments, aptamers are small (15 KD; or between 15-80 nucleotides or between 20-50 nucleotides). Aptamers are generally isolated from libraries consisting of 1014-1015 random oligonucleotide sequences by a procedure termed SELEX (systematic evolution of ligands by exponential enrichment; see, for example, Tuerk et al., Science, 249:505-510, 1990; Green et al., Methods Enzymology. 75-86, 1991; and Gold et al., Annu. Rev. Biochem., 64: 763-797, 1995).

As used herein, the term “binds” refers to an association of a binding domain to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

For instance, LOC 1 410-1 may include a first reagent including a binding ligand with a binding domain that specifically binds a particular analyte-of-interest, as well as a second reagent including an enzyme that outputs a detectable signal (e.g., a color change, a fluorescent signal, etc.) when the enzyme encounters the binding molecule bound to the analyte-of-interest. Example reagents include, for example, lactate peroxidase (e.g., from Sigma-Aldrich of St. Louis, Mo.), pyruvate peroxides, streptavidin-horseradish peroxidase (HRP) (e.g., from ThermoFisher Scientific of Waltham, Mass.), human syndecan-1/CD138 antibody (e.g., from R&D Systems of Minneapolis, Minn.), goat anti-human syndican-1 capture antibody, biotinylated anti-human syndecan-1 detection antibody (e.g., from Bio-Techne Corporation of Minneapolis, Minn.), and the like. Further, LOC 1 410-1 may include a sensor (e.g., a photosensor) configured to detect the signal output by the enzyme. LOC 1 410-1 may be configured to move a sample of the interstitial fluid into one or more reaction chambers, in which the first and second reagents mix with the sample. If present, the analyte-of-interest in the sample binds to the binding molecule and the enzyme. LOC 1 410-1 may transport the resultant mixture to a detection chamber, wherein the sensor is configured to detect the signal output by the enzyme in the detection chamber. In some implementations, the magnitude of the signal is correlated to the concentration of the analyte-of-interest within the detection chamber. Thus, the signal detected by the sensor may be dependent on the presence and/or concentration of the analyte-of-interest in the sample. According to various implementations, the LOCs 1 to n 410-1 to 410-n may each have similar constituent elements, but may respectively detect different analytes-of-interest.

In various cases, the LOCs 1 to n 410-1 to 410-n may include respective sensors configured to detect respective analog signals from respective detection chambers in the LOCs 1 to n 410-1 to 410-n. Furthermore, the LOCs 1 to n 410-1 to 410-n may include one or more analog-to-digital converters (ADCs) configured to generate at least one digital signal based on the analog signals detected by the sensors. In various implementations, the LOCs 1 to n 410-1 to 410-n include and/or are connected to at least one processor and/or integrated circuit (e.g., an application-specific integrated circuit (ASIC)) configured to determine amounts of the analytes in the interstitial fluid based on the digital signal(s), compare the amounts of the analytes to various thresholds, determine whether the amounts of the analytes are consistent with shock, or any combination thereof. In various implementations, the processor(s) and/or integrated circuit(s) are further configured to output indications of the amounts of the analytes and/or whether the analytes are consistent with shock via an output device of the monitoring device. In some cases, the processor(s) and/or integrated circuit(s) are configured to generate a signal indicative of the amounts of the analytes and/or whether the analytes are consistent with shock, and to cause the signal to be transmitted to an external device via a transceiver. Accordingly, the analytes detected by the LOCs 1 to n 410-1 to 410-n can be reported to a user and/or the external device.

The detectors 400 may further include waste receptacles 1 to n 412-1 to 412-n. The waste receptacles 1 to n 412-1 to 412-n may be configured to collect respective waste from the reactions performed in the LOCs 1 to n 410-1 to 410-n. For example, waste receptacle 1 412-1 may receive the mixture of the first reagent, the second reagent, and the interstitial fluid sample from LOC 1 410-1. The waste receptacles 1 to n 412-1 to 412-n may include vials, bags, or any other type of structure that is configured to hold fluids. In some examples, the waste receptacles 1 to n 412-1 to 412-n can be removed and/or emptied manually by a user.

During operation, the detectors 400 may selectively detect analytes in the interstitial fluid of the patient 402 at different sampling rates and/or sampling times. For example, the pump 1 406-1 may transport the perfusate from the perfusate receptacle(s) 404, draw interstitial fluid samples from the patient 402, and the LOC 1 410-1 may detect a first analyte in the interstitial fluid samples at a first sampling rate. In addition, the pump n 406-n may transport the perfusate from the perfusate receptacle(s) 404, draw interstitial fluid samples from the patient 402, and the LOC n 410-n may detect an nth analyte in the interstitial fluid samples at a second sampling rate, wherein the first sampling rate and the second sampling rate are different. In some cases, the LOC 1 410-1 may detect the first analyte in response to a first event (e.g., before a meal of the patient 402) and the LOC n 410-n may detect the second analyte in response to a second event (e.g., two hours after the meal of the patient 402), wherein the first event is different than the second event. Furthermore, in some implementations, the LOC 1 410-1 and the LOC n 410-n may detect their respective analytes using different volumes of interstitial fluid samples drawn from the patient 402.

FIG. 5 illustrates various electronic components within the detectors 400. For example, the electronic components illustrated in FIG. 5 can be included in the same device as the fluidic components illustrated in FIG. 4.

In various implementations, the detectors 400 include a battery 502 configured to power the electronic components within the detectors 400. For example, the battery 502 may provide power to one or more processors 504 in the detectors 400. The processor(s) 504 may be configured to perform various operations. For example, the processor(s) 504 may perform operations stored in memory 506. In various implementations, the processor(s) 504 receive signals (e.g., representing digital data) from other components within the detectors 400. Furthermore, the processor(s) 504 may control other components within the detectors 400 by outputting signals to other components within the detectors 400.

In various implementations, the battery 502 powers one or more electronic valves 508. The electronic valve(s) 508 may be configured to selectively open and close fluidic pathways within the detectors 400. For example, the electronic valve(s) 508 may be disposed in fluidic pathways extending between the perfusate receptacle(s) 404, the pumps 1 to n 406-1 to 406-n, the LOCs 1 to n 410-1 to 410-n, and the waste receptacles 1 to n 412-1 to 412-n. In various cases, the electronic valve(s) 508 are disposed in fluidic pathways within the LOCs 1 to n 410-1 to 410-n. Based on the operation of the electronic valve(s) 508, fluids (e.g., perfusate, samples, reagents, etc.) within the detectors 400 may be directed along certain fluidic pathways and/or may be blocked from other fluidic pathways. In some examples, the electronic valve(s) 508 are individually activated based on signals received from the processor(s) 504.

The detectors 400 also include 1 to n pump actuators 510-1 to 510-n. The pump actuators 510-1 to 510-n may selectively activate the respective pumps 406-1 to 406-n. For example, pump actuator 1 510-1 may activate pump 1 406-1, and so on. The pump actuators 510-1 to 510-n may include motors or other active elements configured to transform electrical energy supplied by the batter 502 into kinetic energy for operating the pumps 406-1 to 406-n. In various implementations, the pump actuators 510-1 to 510-n may be activated by signals received from the processor(s) 504.

Sensors 1 to n 512-1 to 512-n may be incorporated into respective LOCs 410-1 to 410-n. The sensors 1 to n 512-1 to 512-n may be configured to detect signals output by chemical reactions that occurs due to the presence of the analytes in the interstitial fluid samples. For example, the sensors 1 to n 512-1 to 512-n may include photosensors configured to detect optical signals output by enzymes in the LOCs 410-1 to 410-n. In some instances, the sensors 1 to n 512-1 to 512-n include electrical sensors configured to detect electrical signals output by enzymes in the LOCs 410-1 to 410-n. The sensors 512-1 to 512-n, in various implementations, are powered by the battery 502. In some implementations, the sensors 512-1 to 512-n are activated by signals from the processor(s) 504.

In various cases, the detectors 400 also include one or more ADCs 514. The ADC(s) 514 are configured to transform analog signals to digital signals. For example, the sensors 512-1 to 512-n may generate analog signals based on the presence and/or amount of analytes in the interstitial fluid samples. The ADC(s) 514 may convert the analog signals to digital signals and provide the digital signals to the processor(s) 504 for further analysis. Although not specifically illustrated in FIG. 5, in some cases, the detectors 400 include one or more digital to analog converters (DACs) that convert digital signals from the processor(s) 504 into analog signals that are used by the other elements of the detectors 400.

The detectors 400 may also include one or more transceivers 516. The transceiver(s) 516 are configured to transmit and/or receive signals between the detectors 400 and an external device. For example, the transceiver(s) 516 transmit and/or receive signals over one or more communication networks. In various implementations, the processor(s) 504 generate data indicating the presence of the analytes in the interstitial fluid samples, the amount of the analytes in the interstitial fluid samples, whether the analytes in the interstitial fluid samples are indicative of shock, or any combination thereof. The transceiver(s) 516 may transmit at least one signal to an external device that is indicative of the generated data. In some implementations, the transceiver(s) 516 receive at least signal from an external device indicating a sampling frequency, a threshold, an instruction to test for one or more analytes, or a combination thereof. The transceiver(s) 516 may provide data to the processor(s) 504 based on the signal(s) from the external device, which may cause the processor(s) 504 to control the other elements of the detectors 400 accordingly.

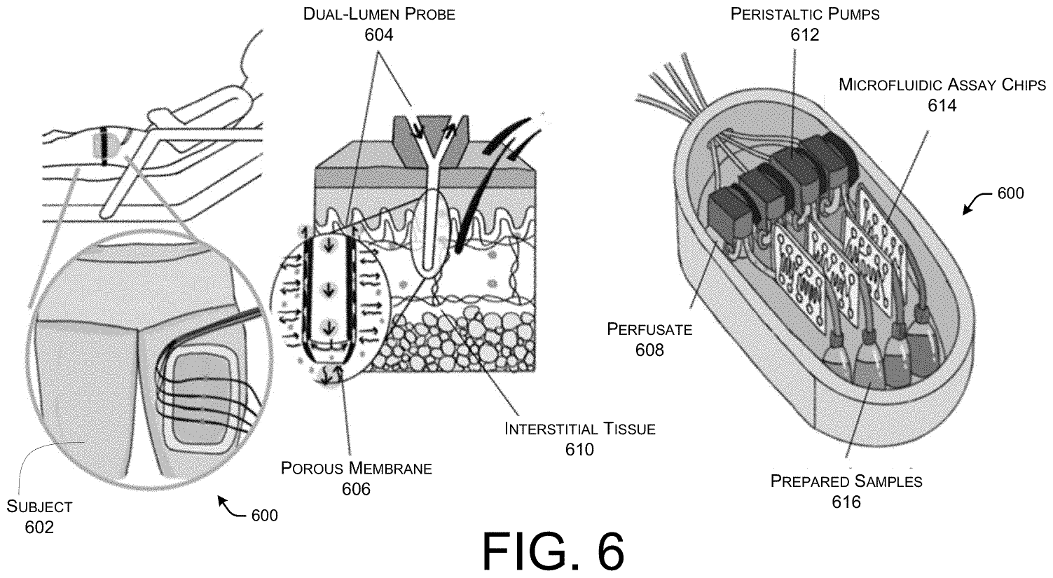

FIG. 6 illustrates an example of a monitoring device 600 configured to be affixed to a thigh of a subject 602. As shown, the monitoring device 600 includes at least one dual-lumen probe 604, which is a form of vertical probe. The dual lumen probe 604 includes a porous membrane 606 through which perfusate 608 is output into interstitial tissue 610 of the subject 602, and through which a sample of interstitial fluid is pulled from the interstitial tissue 610 into the monitoring device 600. The perfusate 608 and interstitial fluid sample are moved via peristaltic pumps 612 within the monitoring device 600.

The peristaltic pumps 612 further move the interstitial fluid sample into microfluidic assay chips 614. The microfluidic assay chips 614 detect multiple analytes in the interstitial fluid sample. In some cases, each microfluidic assay chip 614 detects a different analyte in the interstitial fluid sample. The detected analytes are relevant to whether the patient 602 is experiencing shock. To detect the analytes in the interstitial fluid samples, the microfluidic assay chips may mix the interstitial fluid samples with one or more reagents to produce prepared samples 616. The prepared samples 616 are collected in waste receptacles, which can be removed and/or emptied manually by a user.

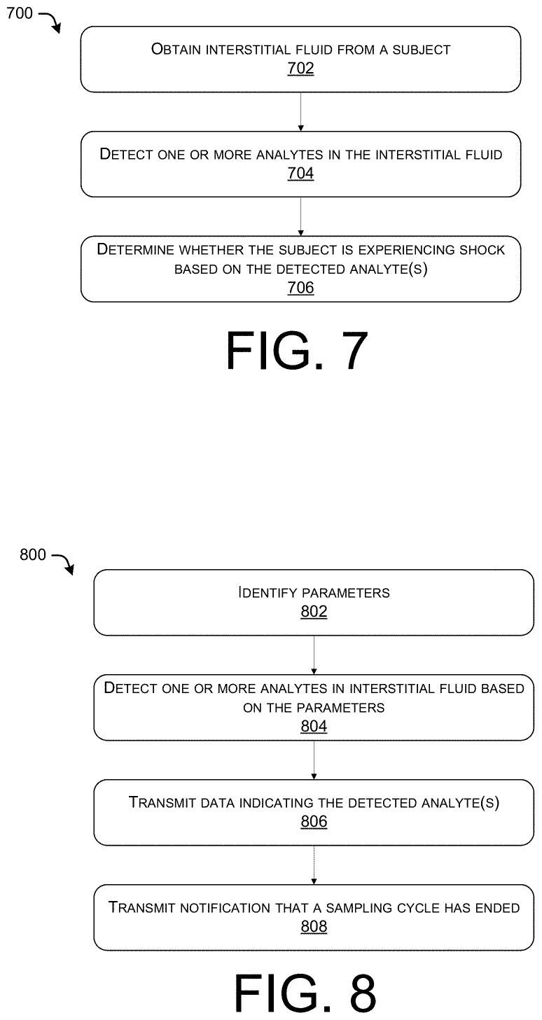

FIG. 7 illustrates an example process 700 for detecting shock in a subject. Process 600 may be performed by an entity, such as the monitoring device 108 described above with reference to FIG. 1 and/or an external computing device.

At 702, the entity obtains interstitial fluid from the subject. In various implementations, the entity obtains the interstitial fluid via at least one probe disposed on the subject. A probe, for example, receives perfusate and outputs a mixture of the interstitial fluid and the perfusate to other components within the device. In various cases, the probe includes a porous structure that separates a sub-epidermal tissue (e.g., an interstitium) of the subject from an interior of the probe. The porous structure includes pores that have widths in a range of 0.1 mm to 0.5 mm. Thus, proteins and other macromolecules from the subject can pass through the porous structure and into the probe. In various implementations, perfusate and interstitial fluid samples are moved through the probe via one or more pumps. In some implementations, the probe is a linear probe or a vertical probe.

A linear probe, for instance, includes a tube that extends through a first insertion site in the skin of the patient, through a sub-epidermal tissue of the patient (e.g., the interstitium), and through a second insertion site in the skin. The porous structure may be at least a portion of the tube of the linear probe. The linear probe is configured to obtain a sample of the interstitial fluid by channeling perfusate through the tube. The perfusate exits the pores in the tube and a mixture of the perfusate and the interstitial fluid enters the pores in the tube. Thus, the tube outputs the sample of the interstitial fluid through the second insertion site.

A vertical probe, in contrast, includes at least one lumen that extends through a single insertion site in the skin of the patient and into the sub-epidermal tissue of the subject. In some examples, the vertical probe is a dual lumen probe including an inner lumen and an outer lumen disposed radially around the inner lumen. A porous structure may be disposed on a distal end of the outer lumen. For instance, the vertical probe is configured to obtain a sample of the interstitial fluid by pushing perfusate through the inner lumen, through the porous structure, and into the sub-epidermal tissue of the subject; and to pull a mixture of the perfusate and interstitial fluid from the sub-epidermal tissue, through the porous structure, and into a space between the inner lumen and the outer lumen. Alternatively, the perfusate may be pushed through the space between the inner lumen and the outer lumen, and the mixture of the perfusate and the interstitial fluid may be pulled up through the inner lumen.

At 704, the entity detects one or more analytes in the interstitial fluid. In various implementations, at least one pump may transport the interstitial fluid (e.g., a mixture of perfusate and interstitial fluid) from the probe(s) to one or more detectors. The detector(s), for instance, may include LOCs configured to detect the analyte(s). In some cases, the LOCs include microfluidic circuits configured to combine the interstitial fluid with one or more reagents. The detectors may include sensors configured to detect signals output by mixtures of the interstitial fluid and the reagent(s). These signals, in some cases, are indicative of the presence and/or amount of the analyte(s) in the interstitial fluid.

In various implementations, the entity detects analyte(s) indicative of shock. For example, the entity detects at least one of lactate, pyruvate, succinate, syndecan-1, syndecan-4, alpha-1-microglobulin, beta-2-microglobulin, cystatin C, retinol-binding protein, albumin, inulin, creatinine, thrombomodulin, e-selectin, intercellular adhesion molecule 1 (ICAM-1), endothelial-leukocyte adhesion molecule 1 (ELAM-1), vascular cell adhesion protein 1 (VCAM-1), interleukin 6 (IL-6), procalcitonin, antithrombin III, protein C, protein S, fibrinogen, tissue plasminogen activator (TPA), alpha 2-antiplasmin, beta-thromboglobulin, platelet factor 4, glucose, sodium, potassium, bicarbonate, chloride, or glycerol. According to some cases, the entity detects multiple analytes associated with different signs of shock, such as a first analyte indicative of tissue hypoxia (e.g., pyruvate and/or lactate) and a second analyte indicative of vascular permeability (e.g., alpha-1 microglobulin and/or syndecan-1). By detecting combination of analytes indicative of different signs of shock, the entity may enhance the accuracy of the determination of whether the subject is in shock.

At 706, the entity determines whether the subject is experiencing shock based on the detected analyte(s). In various implementations, the entity includes an ADC configured to convert analog signals generated by the sensors in the detectors into digital signals. A processor and/or IC (e.g., an ASIC) in the entity may be configured to identify the amount of the analyte(s) in the subject based on the digital signals. Further, the entity may determine whether the subject is in shock based on the amount of the analyte(s). For example, the entity may store a computing model that relates the amount of the analyte(s) to the subject's risk of being in shock, and may determine that the subject is in shock if the risk is greater than a threshold risk. According to some examples, the entity may compare the amount of the analyte(s) to one or more thresholds, and may determine whether the subject is in shock based on the comparison. In some implementations, the computing model may include generating a score based on the amount of multiple analytes detected in the interstitial fluid of the subject. For example, the computing model may add, subtract, divide, and/or multiply multiple analytes and scalar factors in order to generate a single score that can be compared to a single threshold. The entity may determine that the subject is in shock by determining that the score is greater than or less than the single threshold.

In various implementations, the computing model and/or threshold(s) are adjusted based on other conditions of the subject, such as whether the subject has one or more risk factors for shock (e.g., whether the subject has severe burns, has experienced vomiting, whether the subject has been in a traumatic physical accident, etc.) and/or the amount of one or more analytes detected in the blood of the subject. The computing model and/or threshold(s) may be prestored by the entity and/or received from an external device (e.g., as input by a user).

According to various implementations, the entity may output an alert if the entity determines that the subject is in shock. For example, the entity itself may output the alert in a manner discernible to a user (e.g., visually on a screen, audibly using a speaker, etc.). In some cases, the entity may transmit the alert to an external device that outputs the alert to a user.

FIG. 8 illustrates an example process 800 for detecting and reporting at least one analyte in interstitial fluid of a subject. The process 800 may be performed by an entity, such as the monitoring device 108 described above with respect to FIG. 1.

At 802, the entity identifies parameters. In some implementations, the parameters are set remotely.

In some implementations, a monitoring device is installed on a patient. For example, one or more probes are inserted into the patient, the device is placed on the patient, connectors (e.g., Luer connectors) are connected between detectors and probes of the device via tubing, and the device is connected to an internal or external perfusate reservoir.

At 804, the entity detects one or more analytes in the interstitial fluid based on the parameters. For example, the monitoring device begins to sample interstitial fluid from the subject. In some cases, the entity transmits a notification indicating that a sampling cycle has begun.

An initial stage of shock is characterized by hypoxia of cells of the patient. In particular examples, the entity detects at least one analyte-of-interest associated with tissue hypoxia. In some examples, the entity detects one or more markers associated with tissue hypoxia, such as lactate, pyruvate, succinate, or a combination thereof. In various examples, the initial stage of shock is associated with increased vascular permeability, while shock resolution is associated with decreased vascular permeability. In some cases, the device detects one or more markers associated with vascular permeability, such as syndecan-1, syndecan-4, alpha-1-microglobulin, beta-2-microglobulin, cystatin C, retinol-binding protein, albumin, inulin, creatinine, or a combination thereof. In some implementations, the device detects one or more markers associated with inflammation and endothelial integrity, such as soluble thrombomodulin, e-selectin, soluble intercellular adhesion molecule 1 (ICAM-1), soluble endothelial-leukocyte adhesion molecule 1 (ELAM-1), soluble vascular cell adhesion protein 1 (VCAM-1), interleukin 6 (IL-6), procalcitonin, or a combination thereof. In some examples, the device detects one or more markers associated with coagulopathy, such as antithrombin III, protein C, protein S, fibrinogen, tissue plasminogen activator (TPA), alpha 2-antiplasmin, beta-thromboglobulin, platelet factor 4, or a combination thereof. In some cases, the device detects one or more electrolytes or other analytes, such as glucose, sodium, potassium, bicarbonate, chloride, glycerol, or a combination thereof. In some cases, the device detects a marker to be used as an endogenous reference compound, such as urea and/or glucose, from which to calibrate the detected concentrations of other analytes. The presence and/or concentrations of one or more of the analytes-of-interest in interstitial fluid of the patient are associated with whether the patient is experiencing shock, such as an initial or early stage of shock. In some examples, the device determines and/or predicts whether the patient is exhibiting shock, at least in part, by detecting the analyte(s)-of-interest in the interstitial fluid. In addition, the severity of the shock may be inferred based on the concentration of the analyte(s)-of-interest.

At 806, the entity transmits data indicating the detected analyte(s). In some implementations, an external device receives the data and stores the data locally.

At 808, the entity transmits a notification that a sampling cycle has ended. The notification may be output to a user, who may replace waste receptacles, perfusate, and/or other reagents within the monitoring device. Subsequently, a user may reactivate the monitoring device again, or may remove the monitoring device from the subject.

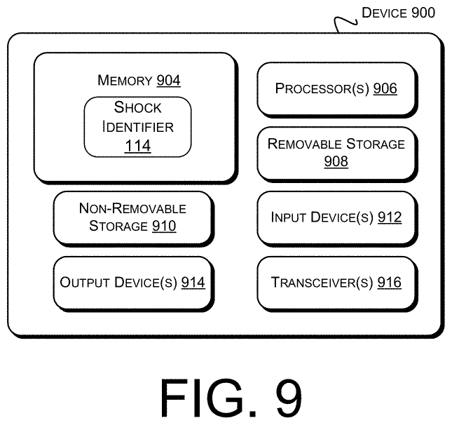

FIG. 9 illustrates an example of a computing device 900 configured to perform various functionality described herein. The computing device 900 may be at least a part of the device described above. The device 900 includes memory 904, at least one processor 906, removable storage 908, non-removable storage 910, at least one input device 912, at least one output device 914, at least one transceiver 916, or a combination thereof. The device 900 may be configured to perform various methods and functions disclosed herein.

The memory 904 may include component(s). The component(s) may include at least one of instruction(s), program(s), database(s), software, operating system(s), etc. In some implementations, the component(s) 918 include instructions that are executed by processor(s) 906 and/or other components of the device 900. For example, the memory 904 may store instructions for executing the shock identifier 114 described above with reference to FIG. 1.

In some implementations, the processor(s) 906 include a central processing unit (CPU), a graphics processing unit (GPU), or both CPU and GPU, or other processing unit or component known in the art.

The device 900 may also include additional data storage devices (removable and/or non-removable) such as, for example, magnetic disks, optical disks, or tape. Such additional storage is illustrated in FIG. 9 by removable storage 908 and non-removable storage 910. Tangible computer-readable media can include volatile and non-volatile, removable and non-removable media implemented in any method or technology for storage of information, such as computer readable instructions, data structures, program modules, or other data. The memory 904, the removable storage 908, and the non-removable storage 910 are all examples of computer-readable storage media. Computer-readable storage media include, but are not limited to, random access memory (RAM), read-only memory (ROM), electrically erasable programmable read-only memory (EEPROM), flash memory, or other memory technology, compact disk read-only memory (CD-ROM), digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the device 900. Any such tangible computer-readable media can be part of the device 900.

The device 900 may be configured to communicate over a network using any common wireless and/or wired network access technology. Moreover, the device 900 may be configured to run any compatible device operating system (OS), including but not limited to, Microsoft Windows Mobile, Google Android, Apple iOS, Linux Mobile, as well as any other common mobile device OS.

The device 900 also can include input device(s) 912, such as a keypad, a cursor control, a touch-sensitive display, voice input device, etc., and output device(s) 914 such as a display, speakers, printers, etc. In some examples, the input device(s) 912 include at least one probe, at least one pump, at least one LOC, at least one sensor, at least one fluid circuit, or a combination thereof.

As illustrated in FIG. 9, the device 900 also includes one or more wired or wireless transceivers 916. For example, the transceiver(s) 916 can include a network interface card (NIC), a network adapter, a local area network (LAN) adapter, or a physical, virtual, or logical address to connect to various network components, for example. To increase throughput when exchanging wireless data, the transceiver(s) 916 can utilize multiple-input/multiple-output (MIMO) technology. The transceiver(s) 916 can include any sort of wireless transceivers capable of engaging in wireless, radio frequency (RF) communication. The transceiver(s) 916 can also include other wireless modems, such as a modem for engaging in Wi-Fi, WiMAX, Bluetooth, infrared communication, and the like. The transceiver(s) 916 may include transmitter(s), receiver(s), or both.

In some examples, the device 900 includes a fluid circuit that is configured to extract interstitial fluid from a patient. The device 900 includes a pump configured to move the interstitial fluid through an LOC in the device 900. The LOC is configured to detect an analyte-of-interest in the interstitial fluid. The processor(s) 906, which is communicatively coupled to the LOC, is configured to generate digital data indicative of the detected analyte-of-interest. In some cases, the output device(s) 914 is configured to output a signal based on the digital data. In some implementations, the transceiver(s) 916 are configured to transmit the digital data to an external device, such as a mobile device (e.g., a mobile phone, tablet computer, laptop computer, etc.), a server, or the like. According to some cases, the processor(s) 906 are configured to generate an alert based on the detected analyte-of-interest as well as a computing model (e.g., a threshold-based model, a trained machine learning model, or the like). The output device(s) 914 output the alert and/or the transceiver(s) 916 transmit the alert to the external device, in some examples. In general, the detected analyte-of-interest is positively correlated to shock, but implementations are not so limited. In various examples, the device 900 is configured to detect multiple analytes-of-interest in the interstitial fluid, and generate the data and/or alert based on the multiple analytes-of-interest.

### Example Clauses

1. A system, including: a monitoring device including: a probe configured to obtain interstitial fluid from a subject; and detectors configured to detect analytes in the interstitial fluid, the analytes including at least one first analyte corresponding to hypoxia of a tissue of the subject and at least one second analyte corresponding to vascular permeability of the subject; and an electronic device configured to output a signal indicative of the detected analytes.
2. The system of clause 1, wherein the at least one first analyte includes pyruvate and/or lactate.
3. The system of clause 1 or 2, wherein the at least one second analyte includes alpha-1 microglobulin and/or syndecan-1.
4. The system of any one of clauses 1 to 3, wherein the at least one first analyte and the at least one second analyte include at least one of lactate, pyruvate, succinate, syndecan-1, syndecan-4, alpha-1-microglobulin, beta-2-microglobulin, cystatin C, retinol-binding protein, albumin, inulin, creatinine, thrombomodulin, e-selectin, intercellular adhesion molecule 1 (ICAM-1), endothelial-leukocyte adhesion molecule 1 (ELAM-1), vascular cell adhesion protein 1 (VCAM-1), interleukin 6 (IL-6), procalcitonin, antithrombin III, protein C, protein S, fibrinogen, tissue plasminogen activator (TPA), alpha 2-antiplasmin, beta-thromboglobulin, platelet factor 4, glucose, sodium, potassium, bicarbonate, chloride, or glycerol.
5. The system of any one of clauses 1 to 4, further including: an analog-to-digital converter configured to generate data indicative of the detected analytes; a processor; memory storing instructions that, when executed by the processor, cause the processor to perform operations including: determining whether the subject is in shock based on a computing model and the data; and generating an alert based on whether the subject is in shock; and a transceiver configured to transmit the alert and/or the data over a wired and/or wireless interface.
6. The system of clause 5, wherein the electronic device includes a display device configured to receive the data and to output a graphical element indicative of the alert and/or the data.
7. The system of clause 5 or 6, wherein the probe includes: an inlet receiving the interstitial fluid from the subject; an outlet fluidly connected to the detectors; and a porous structure disposed between the inlet and the outlet.
8. The system of clause 7, further including: a receptacle storing perfusate; a pump configured to push the perfusate into the probe and to pull a mixture of the perfusate and the interstitial fluid from the probe through the outlet.
9. The system of any one of clauses 5 to 8, further including: one or more pumps configured to cause the interstitial fluid to flow from the probe to the detectors; and one or more tubes fluidly connecting the probe and the detectors.
10. The system of clause 9, wherein the operations further include activating the one or more pumps.
11. The system of clause 10, wherein the operations further include receiving an input signal, and wherein activating the one or more pumps is based on receiving the input signal.
12. The system of any one of clauses 5 to 11, wherein the probe includes a linear probe at least partially disposed underneath the skin of the subject, the linear probe extending through a first insertion site in the skin, through an interstitial space of the subject, and through a second insertion site in the skin.
13. The system of any one of clauses 5 to 12, wherein the probe includes a vertical probe at least partially disposed underneath the skin of the subject, the vertical probe extending into an interstitial space of the subject from a single insertion site in the skin of the subject.
14. The system of any one of clauses 5 to 13, wherein the detectors include labs-on-chips (LOCs).
15. The system of clause 14, wherein at least one of the LOCs includes: a microfluidic circuit configured to carry the interstitial fluid and one or more reagents; one or more receptacles configured to store the one or more reagents; and one or more sensors configured to detect an analyte in the interstitial fluid based on a mixture of the interstitial fluid and the one or more reagents.
16. The system of clause 15, wherein the one or more reagents include a binding ligand (e.g., an antibody or binding fragment thereof) configured to bind to the analyte, and wherein the one or more sensors are configured to detect the binding ligand bound to the analyte.
17. The system of any one of clauses 5 to 16, wherein determining whether the subject is in shock based on the computing model and the data includes: determining levels of the analytes in the interstitial fluid based on the data; and comparing the levels of the analytes to one or more thresholds.
18. The system of any one of clauses 6 to 17, wherein the display device includes a mobile device.
19. A medical device, including: a probe configured to obtain interstitial fluid from a subject; and detectors configured to detect analytes in the interstitial fluid.
20. The medical device of clause 19, wherein the probe includes: an inlet receiving the interstitial fluid from the subject; an outlet fluidly connected to the detectors; and a porous structure disposed between the inlet and the outlet.
21. The medical device of clause 20, further including: a receptacle storing perfusate; a pump configured to push the perfusate into the probe and to pull a mixture of the perfusate and the interstitial fluid from the probe through the outlet.
22. The medical device of any one of clauses 19 to 21, further including: one or more pumps configured to cause the interstitial fluid to flow from the probe to the detectors; and one or more tubes fluidly connecting the probe and the detectors.
23. The medical device of any one of clauses 19 to 22, wherein the probe includes a linear probe at least partially disposed underneath the skin of the subject, the linear probe extending through a first insertion site in the skin, through an interstitial space of the subject, and through a second insertion site in the skin.
24. The medical device of any one of clauses 19 to 23, wherein the probe includes a vertical probe at least partially disposed underneath the skin of the subject, the vertical probe extending into an interstitial space of the subject from a single insertion site in the skin of the subject.
25. The medical device of any one of clauses 19 to 24, wherein the detectors include one or more labs-on-chips (LOCs).
26. The medical device of clause 25, wherein at least one of the LOCs includes: a microfluidic circuit configured to carry the interstitial fluid and one or more reagents; one or more receptacles configured to store the one or more reagents; and one or more sensors configured to detect an analyte in the interstitial fluid based on a mixture of the interstitial fluid and the one or more reagents.
27. The medical device of clause 26, wherein the one or more reagents include a binding ligand (e.g., an antibody or binding fragment thereof) configured to bind to the analyte, and wherein the one or more sensors are configured to detect the binding ligand bound to the analyte.
28. The medical device of any one of clauses 19 to 27, wherein the analytes include pyruvate and/or lactate.
29. The medical device of any one of clauses 19 to 28, wherein the analytes include alpha-1 microglobulin and/or syndecan-1.
30. The medical device of any one of clauses 19 to 29, wherein the analytes include at least one of lactate, pyruvate, succinate, syndecan-1, syndecan-4, alpha-1-microglobulin, beta-2-microglobulin, cystatin C, retinol-binding protein, albumin, inulin, creatinine, thrombomodulin, e-selectin, intercellular adhesion molecule 1 (ICAM-1), endothelial-leukocyte adhesion molecule 1 (ELAM-1), vascular cell adhesion protein 1 (VCAM-1), interleukin 6 (IL-6), procalcitonin, antithrombin III, protein C, protein S, fibrinogen, tissue plasminogen activator (TPA), alpha 2-antiplasmin, beta-thromboglobulin, platelet factor 4, glucose, sodium, potassium, bicarbonate, chloride, glycerol, or urea.
31. The medical device of any one of clauses 19 to 30, further including: an analog-to-digital converter configured to generate data indicative of the detected analytes; a processor; and memory storing instructions that, when executed by the processor, cause the processor to perform operations including: determining that the subject is in shock based on a computing model and the data; and generating an alert based on determining that the subject is in shock.
32. The medical device of clause 31, further including a transceiver configured to transmit the alert and/or the data over a wired and/or wireless interface.
33. The medical device of clause 31 or 32, wherein determining whether the subject is in shock based on the computing model and the data includes: determining levels of the analytes in the interstitial fluid based on the data; and comparing the levels of the analytes to one or more thresholds.
34. A method, including: obtaining interstitial fluid from a subject; and detecting analytes in the interstitial fluid, the analytes including at least one first analyte corresponding to hypoxia of a tissue of the subject and at least one second analyte corresponding to vascular permeability of the subject.
35. The method of clause 34, further including: determining whether the subject is in shock based on a computing model and the detected analytes; and generating an alert based on whether the subject is in shock.
36. The method of clause 35, further including: transmitting the alert over a wired and/or wireless interface.
37. The method of clause 36, further including: receiving an input signal; and obtaining the interstitial fluid based on the input signal.
38. The method of clause 37, wherein determining whether the subject is in shock based on the computing model and the data includes: determining levels of the analytes in the interstitial fluid based on the data; and comparing the levels of the analytes to one or more thresholds.
39. A device as substantially described herein.
40. A system as substantially described herein.
41. A method as substantially described herein.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing the invention in diverse forms thereof. This disclosure references various articles, books, patent documents, and references, each of which is incorporated by reference herein in its entirety.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, an example of a material effect corresponds to any reduction in the accuracy of the device to detect and/or predict shock and/or the portability of the device. As used herein, the term “based on” is equivalent to “based at least partly on,” unless otherwise specified.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person ordinarily skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or implementations of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations of this invention are described herein, including the best mode known to the inventors for carrying out the invention. Of course, variations on these described implementations will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the implementations disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred implementations of the present invention only and are presented in the cause of providing what is believed to be the most useful and readily understood description of the principles and conceptual aspects of various implementations of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A system, comprising:
a monitoring device comprising:
at least one receptacle storing perfusate;
at least one probe configured to receive the perfusate and to obtain interstitial fluid from a subject; and
detectors configured to detect analytes in the interstitial fluid, the analytes comprising at least one first analyte corresponding to hypoxia of a tissue of the subject and at least one second analyte corresponding to vascular permeability of the subject;
at least one pump configured to move the perfusate into the at least one probe and to move the interstitial fluid from the at least one probe into the detectors;
a processor configured to determine that the subject is in shock based on the detected analytes; and
a transceiver configured to transmit a first signal indicating that the subject is in shock; and
an electronic device configured to receive the first signal and to output a second signal indicating that the subject is in shock.

2. The system of claim 1, wherein the at least one first analyte comprises pyruvate and/or lactate, and wherein the at least one second analyte comprises alpha-1 microglobulin and/or syndecan-1.

3. The system of claim 1, wherein the at least one first analyte and the at least one second analyte comprise at least one of lactate, pyruvate, succinate, syndecan-1, syndecan-4, alpha-1-microglobulin, beta-2-microglobulin, cystatin C, retinol-binding protein, albumin, inulin, creatinine, thrombomodulin, e-selectin, intercellular adhesion molecule 1 (ICAM-1), endothelial-leukocyte adhesion molecule 1 (ELAM-1), vascular cell adhesion protein 1 (VCAM-1), interleukin 6 (IL-6), procalcitonin, antithrombin III, protein C, protein S, fibrinogen, tissue plasminogen activator (TPA), alpha 2-antiplasmin, beta-thromboglobulin, platelet factor 4, sodium, potassium, bicarbonate, chloride, or glycerol.

4. The system of claim 1, wherein the at least one probe comprises a linear probe at least partially disposed underneath the skin of the subject, the linear probe extending through a first insertion site in the skin, through an interstitial space of the subject, and through a second insertion site in the skin, or
wherein the at least one probe comprises a vertical probe at least partially disposed underneath the skin of the subject, the vertical probe extending into an interstitial space of the subject from a single insertion site in the skin of the subject.

5. The system of claim 1, wherein the detectors comprise labs-on-chips (LOCs) comprising:
a microfluidic circuit configured to carry the interstitial fluid and one or more reagents;
one or more receptacles configured to store the one or more reagents; and
one or more sensors configured to detect the first analyte or the second analyte in the interstitial fluid based on a mixture of the interstitial fluid and the one or more reagents.

6. The system of claim 1, wherein determining that the subject is in shock based on the detected analytes comprises
comparing the amounts of the detected analytes to one or more thresholds.

7. The system of claim 1, wherein the detectors comprise a first detector configured to detect the first analyte and a second detector configured to detect the second analyte,
wherein the at least one pump is configured to move the interstitial fluid into the first detector at a first flow rate and/or a first sampling rate and to move the interstitial fluid into the second detector at a second flow rate and/or a second sampling rate, and
wherein the first flow rate is different than the second flow rate and/or the first sampling rate is different than the second sampling rate.

8. A medical device, comprising:
at least one probe configured to obtain interstitial fluid from a subject; and
a first detector configured to detect an amount of a first analyte in the interstitial fluid, the first analyte being indicative of hypoxia in the subject;
a second detector configured to detect an amount of a second analyte in the interstitial fluid, the second analyte being indicative of vascular permeability of the subject; and
a processor configured to determine whether the subject is in shock based on the amount of the first analyte and the amount of the second analyte in the interstitial fluid.

9. The medical device of claim 8, wherein the at least one probe comprises:
an inlet configured to receive perfusate;
an outlet configured to output a mixture of the perfusate and the interstitial fluid to the first detector and/or the second detector; and
a porous structure disposed between the inlet and the outlet, and
wherein the medical device further comprises:
a receptacle storing the perfusate; and
at least one pump configured to push the perfusate into the inlet of the at least one probe and to pull the mixture of the perfusate and the interstitial fluid from the at least one probe.

10. The medical device of claim 8, wherein the at least one probe comprises a linear probe at least partially disposed underneath the skin of the subject, the linear probe extending through a first insertion site in the skin, through an interstitial space of the subject, and through a second insertion site in the skin.

11. The medical device of claim 8, wherein the at least one probe comprises a vertical probe at least partially disposed underneath the skin of the subject, the vertical probe extending into an interstitial space of the subject from a single insertion site in the skin of the subject.

12. The medical device of claim 8, wherein the first detector or the second detector comprises:
a microfluidic circuit configured to carry the interstitial fluid and one or more reagents;
one or more receptacles configured to store the one or more reagents; and
one or more sensors configured to detect an analyte in the interstitial fluid based on a mixture of the interstitial fluid and the one or more reagents,
wherein the one or more reagents comprise a binding ligand that binds the analyte, and
wherein the one or more sensors are configured to detect the binding ligand bound to the analyte.

13. The medical device of claim 8, wherein the first analyte comprises pyruvate and/or lactate, and
wherein the second analyte comprises alpha-1 microglobulin and/or syndecan-1.

14. The medical device of claim 8, wherein the first analyte and the second analyte comprise at least one of lactate, pyruvate, succinate, syndecan-1, syndecan-4, alpha-1-microglobulin, beta-2-microglobulin, cystatin C, retinol-binding protein, albumin, inulin, creatinine, thrombomodulin, e-selectin, intercellular adhesion molecule 1 (ICAM-1), endothelial-leukocyte adhesion molecule 1 (ELAM-1), vascular cell adhesion protein 1 (VCAM-1), interleukin 6 (IL-6), procalcitonin, antithrombin III, protein C, protein S, fibrinogen, tissue plasminogen activator (TPA), alpha 2-antiplasmin, beta-thromboglobulin, platelet factor 4, sodium, potassium, bicarbonate, chloride, or glycerol.

15. The medical device of claim 8, further comprising:
an analog-to-digital converter configured to generate data indicative of the detected analytes;
a processor; and
memory storing instructions that, when executed by the processor, cause the processor to perform operations comprising:
determining that the subject is in shock based on a computing model and the data;
generating an alert based on determining that the subject is in shock; and
outputting the alert.

16. The medical device of claim 15, further comprising a transceiver configured to transmit the alert and/or the data over a wired and/or wireless interface.

17. The medical device of claim 15, wherein determining whether the subject is in shock based on the computing model and the data comprises:
determining levels of the analytes in the interstitial fluid based on the data; and
comparing the levels of the analytes to one or more thresholds.

18. A method, comprising:
obtaining interstitial fluid from a subject; and
detecting analytes in the interstitial fluid, the analytes comprising at least one first analyte corresponding to hypoxia of a tissue of the subject and at least one second analyte corresponding to vascular permeability of the subject
determining whether the subject is in shock based on a computing model and the detected analytes; and
generating an alert based on whether the subject is in shock.

19. The method of claim 18, further comprising:
transmitting the alert over a wired and/or wireless interface.

20. The method of claim 18, wherein determining whether the subject is in shock based on the computing model and the data comprises:
determining levels of the analytes in the interstitial fluid based on the data; and
comparing the levels of the analytes to one or more thresholds.
